SEQUENCE LISTING (18 sequences)

SEQ ID NO: 1 - protein, 311 aa, Xanthobacter autotrophicus
MINAIRTPDQRFSNLDQYPFSPNYLDDLPGYPGLRAHYLDEGNSDAEDVFLCLHGEPTWSYLYRKMIPVFAESGARVIAPDFFGFGKSDKPVDEEDYTFEFHRNFLLALIERLDLRNITLVVQDWGGFLGLTLPMADPSRFKRLIIMNACLMTDPVTQPAFSAFVTQPADGFTAWKYDLVTPSDLRLDQFMKRWAPTLTEAEASAYAAPFPDTSYQAGVRKFPKMVAQRDQACIDISTEAISFWQNDWNGQTFMAIGMKDKLLGPDVMYPMKALINGCPEPLEIADAGHFVQEFGEQVAREALKHFAETEZ

SEQ ID NO: 2 - DNA, 933 bp, Xanthobacter autotrophicus
atgataaatgcaattcgcaccccggaccaacgcttcagcaatctcgatcagtatccgttcagccccaactacctggacgacctccccggctacccgggattgcgggcacactacctcgacgagggcaattctgacgctgaagacgtttttctctgccttcatggcgagcccacctggagttacctgtatcgcaagatgatcccggtatttgctgaatcaggcgcacgagttattgcgccagacttttttggattcggaaaatccgacaagccagtagacgaagaagactacaccttcgaatttcaccgcaacttcctgcttgcactaatcgaacggcttgacttgcgcaacattacgctggtcgttcaggactggggcggatttttggggctgaccttaccgatggccgacccttcccgcttcaagcgcctgatcatcatgaacgcctgcttgatgaccgacccggtcacccagcctgcgtttagcgcctttgtcacccagcctgcggatggctttaccgcctggaaatacgatctggttacgccatcagacctgcgccttgaccagttcatgaagcgttgggcgcccacactgaccgaagctgaggcctccgcgtatgctgcgcctttccctgacacttcctatcaggctggtgtacgcaagtttcccaagatggtcgcgcaacgcgaccaggcctgcatcgacatttcaaccgaagcgatttcgttctggcagaacgactggaatggccagaccttcatggccattggcatgaaagacaaattgctgggaccggacgtcatgtatcctatgaaggcgctcattaatggctgcccggaacccctcgaaatagcggacgctggccatttcgtacaggagtttggcgagcaagtggctcgcgaggccctgaaacactttgccgagacagaatag

SEQ ID NO: 3 - protein, 236 aa, Artificial Sequence
  Synthetic BC 3334 N122Y
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITYGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 4 - DNA, 711 bp, Artificial Sequence
  Synthetic BC 3334 N122Y gene
atgaaatacaaagttatattattcgacgtagatgatacattattagatttccctgaaacggaaagacacgcattacataatgcgtttgtacagtttgatatgcctacagggtataatgattatcttgcaagctataaagagattagtaatggattatggagagatttagaaaataaaatgattacgctaagtgaattagcagtagatcgatttagacaattatttgcacttcataatatagacgtagatgcacagcaatttagtgatgtataccttgaaaatttagggaaggaagtacatcttatagaaggcgcagtacaattatgtgaaaatctacaagattgcaagttaggtattattacgtatggatatacgaaggtgcaacaatcaagaatcggaaattcacctttatgtaatttctttgatcacattattatttctgaagaagttggtcatcaaaaaccagcacgtgagatttttgattatgcgtttgagaagtttgggattactgataaatcaagcgtactaatggttggagattcgttaacttctgatatgaaaggcggagaagattacggcattgatacgtgttggtataatccgagtttgaaagaaaacgggacagatgttaacccgacttatgaagtggagagtctgctccaaattttagaaattgtagaagtggcggaagaaaaggtagcttcattttaa

SEQ ID NO: 5 - protein, 247 aa, Pseudomonas saitens
MEKVKAILFDKDGTLMDFHSIWIKVAEELVAECISLYHLPSTIGQTLLEEIGVEGAFVNPRSAIAAGTSLDVAKGLCNYIESAREEEMHQWVSEKLFSLMYEHRSHMKMTADLPKVLQALKDKGFILGVVTADDFAPTELFLKQYKLENFFDYIIASDTFPAQKPDKKIIEVFCEKFNLESCEVAVVGDTPTDLHLAKNGDCYAIGVLSGTGDRPTLEPLADLVLDSVGEFISQSGEFFWEKEKSNV

SEQ ID NO: 6 - DNA, 744 bp, Pseudomonas saitens
atggaaaaagttaaagcgatcctgttcgataaagatggcaccctgatggatttccacagcatctggatcaaagttgcggaagaactggttgcggaatgcatcagcctgtaccacctgccgtccaccatcggtcagaccctgctggaagaaatcggcgttgaaggcgcgttcgttaacccgcgtagcgcgatcgcggcgggcaccagcctggatgttgcgaaaggcctgtgcaactacatcgaaagcgcgcgtgaagaagaaatgcaccagtgggttagcgaaaaactgttcagcctgatgtacgaacaccgtagccacatgaaaatgaccgcggatctgccgaaagttctgcaggctctgaaagataaaggtttcatcctgggcgttgttaccgcggatgatttcgcgccgaccgaactgttcctgaaacagtacaaactggaaaacttcttcgattacatcatcgcgagcgataccttcccggcacagaaaccggataagaaaatcatcgaagtgttctgcgaaaaattcaacctggaaagctgtgaagttgcggttgttggcgataccccgaccgatctgcacctggcgaaaaacggtgattgctacgcgatcggtgttctgtccggtaccggtgatcgtccgaccctggaaccgctggcggatctggttctggattctgttggtgaattcatcagccagagcggtgaattcttctgggaaaaagaaaaaagcaacgtttaa

SEQ ID NO: 7 - DNA, 33 bp, Artificial Sequence
  Synthetic primer
aaggaagctaaaatgatcaacgcaatccgcacc

SEQ ID NO: 8 - DNA, 36 bp, Artificial Sequence
  Synthetic primer
taaaaaaattacgccttattcggtttccgcaaagtg

SEQ ID NO: 9 - DNA, 36 bp, Artificial Sequence
  Synthetic primer
aaggaagctaaaatgaaccacaaatacacctgcccg

SEQ ID NO: 10 - DNA, 43 bp, Artificial Sequence
  Synthetic primer
taaaaaaattacgccttacacattagacttttctttctcccag

SEQ ID NO: 11 - DNA, 40 bp, Artificial Sequence
  Synthetic primer
aagaaggagatataccatgaaatacaaagttatattattc

SEQ ID NO: 12 - DNA, 41 bp, Artificial Sequence
  Synthetic primer
gcattatgcggccgcaagctttaaaatgaagctaccttttc

SEQ ID NO: 13 - DNA, 43 bp, Artificial Sequence
  Synthetic primer
aaggaagctaaaatgaaatacaaagttatattattcgacgtag

SEQ ID NO: 14 - DNA, 41 bp, Artificial Sequence
  Synthetic primer
taaaaaaattacgccttaaaatgaagctaccttttcttccg

SEQ ID NO: 15 - DNA, 23 bp, Artificial Sequence
  Synthetic primer
cattttagcttccttagctcctg

SEQ ID NO: 16 - DNA, 21 bp, Artificial Sequence
  Synthetic primer
ggcgtaatttttttaaggcag

SEQ ID NO: 17 - protein, 236 aa, Bacillus cereus
MKYKVILFDVDDTLLDFPETERHALHNAFVQFDMPTGYNDYLASYKEISNGLWRDLENKMITLSELAVDRFRQLFALHNIDVDAQQFSDVYLENLGKEVHLIEGAVQLCENLQDCKLGIITNGYTKVQQSRIGNSPLCNFFDHIIISEEVGHQKPAREIFDYAFEKFGITDKSSVLMVGDSLTSDMKGGEDYGIDTCWYNPSLKENGTDVNPTYEVESLLQILEIVEVAEEKVASF

SEQ ID NO: 18 - DNA, 711 bp, Bacillus cereus
atgaaatacaaagttatattattcgacgtagatgatacattattagatttccctgaaacggaaagacacgcattacataatgcgtttgtacagtttgatatgcctacagggtataatgattatcttgcaagctataaagagattagtaatggattatggagagatttagaaaataaaatgattacgctaagtgaattagcagtagatcgatttagacaattatttgcacttcataatatagacgtagatgcacagcaatttagtgatgtataccttgaaaatttagggaaggaagtacatcttatagaaggcgcagtacaattatgtgaaaatctacaagattgcaagttaggtattattacgaatggatatacgaaggtgcaacaatcaagaatcggaaattcacctttatgtaatttctttgatcacattattatttctgaagaagttggtcatcaaaaaccagcacgtgagatttttgattatgcgtttgagaagtttgggattactgataaatcaagcgtactaatggttggagattcgttaacttctgatatgaaaggcggagaagattacggcattgatacgtgttggtataatccgagtttgaaagaaaacgggacagatgttaacccgacttatgaagtggagagtctgctccaaattttagaaattgtagaagtggcggaagaaaaggtagcttcattttaa

(19) United States
(12) Patent Application Publication
(10) Pub. No.: US 2019/0032034 A1
(43) Pub. Date: Jan. 31, 2019

(54) MICROORGANISM INCLUDING GENE ENCODING PROTEIN HAVING DEHALOGENASE ACTIVITY, AND METHOD OF REDUCING CONCENTRATION OF FLUORINE-CONTAINING COMPOUND IN SAMPLE USING THE SAME

(71) Applicant: Samsung Electronics Co., Ltd., Suwon-si (KR)

(72) Inventors: Jinha Kim, Namyangju-si (KR); Jongwon Byun, Suwon -si (KR); Jinhwan Park, Suwon-si (KR)

(21) Appl. No.: 16/046,685

(22) Filed: Jul. 26, 2018

(30) Foreign Application Priority Data
Jul. 28, 2017 (KR) 10-2017-0096385

(51) Int. Cl. C12N 9/14; C02F 3/34; B01D 53/84; B01D 53/70

(52) U.S. Cl. CPC C12N 9/14; C12Y 308/01002; C02F 2101/36; B01D 53/84; B01D 53/70; C02F 3/342

(57) ABSTRACT

Provided are a microorganism including a gene encoding a protein having a dehalogenase activity, a composition including the microorganism for use in reducing a concentration of a fluorine-containing compound in a sample, and a method of reducing the concentration of the fluorine-containing compound in the sample by using the microorganism.

### CROSS-REFERENCE TO RELATED APPLICATION

This application claims the benefit of Korean Patent Application No. 10-2017-0096385, filed on Jul. 28, 2017, in the Korean Intellectual Property Office, the disclosure of which is incorporated herein in its entirety by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 15,342 Byte ASCII (Text) file named “739335_ST25.TXT,” created on Jul. 26, 2018.

### BACKGROUND

### 1. Field

The present disclosure relates to a microorganism including a gene encoding a protein having a dehalogenase activity, a composition including the microorganism for use in reducing a concentration of a fluorine-containing compound in a sample, and a method of reducing a concentration of a fluorine-containing compound in a sample.

### 2. Description of the Related Art

The emissions of greenhouse gases which have accelerated global warming are serious environmental problems, and regulations to reduce and prevent the emissions of greenhouse gases have been tightened. Among the greenhouse gases, fluorinated gases (F-gases), such as perfluorocarbons (PFCs), hydrofluorocarbon (HFCs), or sulfur hexafluoride (SF₆), show low absolute emission, but have a long half-life and a very high global warming potential, resulting in significantly adverse environmental impacts. The amount of F-gases emitted from semiconductor and electronics industries, which are major causes of F-gas emission, has exceeded the assigned amount of greenhouse gas emissions and continues to increase. Therefore, costs required for decomposition of greenhouse gases and greenhouse gas emission allowances are increasing every year.

A pyrolysis or catalytic thermal oxidation process has been generally used in the decomposition of F-gases. However, such a process has disadvantages of limited decomposition rate, emission of secondary pollutants, high cost, etc. To overcome these disadvantages, biological decomposition of F-gases using a microbial biocatalyst has been proposed. Accordingly, it is expected to overcome limitations of the chemical decomposition process and also to treat F-gases in a more economical and environmentally-friendly manner.

A dehalogenase is a type of a hydroxylase, but the catalytic ability of a dehalogenase for the removal of a fluorine atom from a substrate is not known in the art.

Despite the efforts of the prior art, there remains a need for a microorganism including a dehalogenase gene which acts on a fluorine-containing compound, a composition including the microorganism and which is capable of reducing a concentration of a fluorine-containing compound in a sample, and a method of reducing a concentration of a fluorine-containing compound in a sample.

### SUMMARY

Provided is a recombinant Pseudomonas saitens microorganism having a genetic modification that increases dehalogenase activity as compared with a parent strain.

Provided is a composition including the recombinant microorganism for use in reducing a concentration of a fluorine-containing compound in a sample, wherein the fluorine-containing compound is represented by Formula 1 or Formula 2.

Provided is a method of reducing a concentration of a fluorine-containing compound in a sample, the method including contacting the recombinant microorganism with a sample, so as to reduce the concentration of the fluorine-containing compound in the sample.

### BRIEF DESCRIPTION OF THE DRAWINGS

These and/or other aspects will become apparent and more readily appreciated from the following description of the embodiments, taken in conjunction with the accompanying drawings in which:

FIG. 1 is a vector map of a pET-BC334 vector; and

FIG. 2 is a schematic diagram of a glass Dimroth reflux condenser.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items.

The term “increase in activity”, “increased activity”, or “increases activity” as used herein may refer to a detectable increase in an activity of a cell, a protein, or an enzyme. The term “increase in activity” or “increased activity” as used herein may also refer to an activity level of a modified (e.g., genetically engineered) cell, protein, or enzyme that is higher than that of a comparative cell, protein, or enzyme of the same type, such as a cell, protein, or enzyme that does not have a given genetic modification (e.g., original or “wild-type” cell, protein, or enzyme). The term “activity of a cell” as used herein may refer to an activity of a particular protein or enzyme of a cell. For example, an activity of a modified or engineered cell, protein, or enzyme may be increased by about 5% or more, about 10% or more, about 15% or more, about 20% or more, about 30% or more, about 50% or more, about 60% or more, about 70% or more, or about 100% or more relative to an activity of a non-engineered cell, protein, or enzyme of the same type, i.e., a wild-type cell, protein, or enzyme. A cell having an increased activity of a protein or an enzyme may be identified by using any method known in the art.

An increase in an activity of an enzyme or a polypeptide may be achieved by an increase in expression or specific activity. The increase in expression may be caused by introduction of a polynucleotide encoding the enzyme or the polypeptide into a cell; by otherwise increasing of the copy number of a gene encoding an enzyme or a polypeptide, by modification of a regulatory region of a polynucleotide encoding the enzyme or the polypeptide. A microorganism into which the gene is introduced may be a microorganism endogenously including the gene (e.g., the exogenous gene is homologous to the microorganism), or a microorganism that does not endogenously include the gene (e.g., the exogenous gene is heterologous to the microorganism). The gene may be operably linked to a regulatory sequence that allows expression thereof, for example, a promoter, an enhancer, a polyadenylation site, or a combination thereof. The polynucleotide whose copy number is increased may be endogenous or exogenous. An endogenous gene is a gene that exists in the genetic material of a microorganism prior to the genetic modification that increases the activity of the enzyme or polypeptide. An exogenous gene refers to a gene that is introduced into a cell in the genetic modification, and may be homologous or heterologous with respect to a host cell into which the gene is introduced. The term “heterologous” means “foreign” or “not native.” Thus, an “exogenous” gene can be introduced despite the preexistence of the same or similar gene in the microorganism.

An “increase of the copy number” may be caused by introduction of an exogenous gene or amplification of an endogenous gene. Optionally, the increase in copy number achieved by genetically engineering a cell to introduce a gene that does not exist in a non-engineered cell. The introduction of the gene may be mediated by a vehicle such as a vector. The introduction may be via a transient introduction in which the gene is not integrated into a genome (e.g., in an episome such as a plasmid), or via integration of the gene into the genome. The introduction may be performed, for example, by introducing a vector into the cell, the vector including a polynucleotide encoding a target polypeptide, and then, replicating the vector in the cell, or by integrating the polynucleotide into the genome.

The introduction of the gene may be performed via a known method, for example, transformation, transfection, or electroporation. The gene may be introduced via a vehicle or as it is. The term “vehicle”, as used herein, refers to a nucleic acid molecule that is able to deliver other nucleic acids linked thereto. As a nucleic acid sequence mediating introduction of a specific gene, the vehicle used herein is construed to be interchangeable with a vector, a nucleic acid construct, and a cassette. The vector may include, for example, a plasmid vector, a virus-derived vector, etc. The plasmid can be a circular double-stranded DNA molecule linkable with another DNA. The vector may include, for example, a plasmid expression vector, a virus expression vector, such as a replication-defective retrovirus, adenovirus, adeno-associated virus, or a combination thereof.

The genetic modification used in the present disclosure may be performed by any molecular biological method known in the art.

The term “parent cell” refers to an original cell or a cell of the same type as an original cell from which a genetically engineered cell is produced. With respect to a particular genetic modification, the “parent cell” may be a cell (whether previously engineered or not) that lacks the particular genetic modification, but is identical in all other respects. Thus, the parent cell may be a cell that is used as a starting material to produce a genetically engineered microorganism having increased activity of a given protein (e.g., a protein having a sequence identity of about 85% or higher amino acid identity with respect to haloalkane dehalogenase). The same comparison is also applied to other genetic modifications. In some embodiments, the parent cell can be a wild-type cell.

The term “gene”, as used herein, refers to a polynucleotide or nucleotide fragment which may express a particular protein, and which may or may not be operably linked to a regulatory sequence (e.g, a 5′-non coding sequence and/or a 3′-non coding sequence).

The term “sequence identity” of a polynucleotide or a polypeptide, as used herein, refers to a degree of identity between bases or amino acid residues of sequences obtained after the sequences are aligned so as to best match in certain comparable regions. The sequence identity is a value that is measured by comparing two sequences in certain comparable regions via optimal alignment of the two sequences, in which portions of the sequences in the certain comparable regions may be added or deleted compared to reference sequences. A percentage of sequence identity may be calculated by, for example, comparing two optimally aligned sequences in the entire comparable regions, determining the number of locations in which the same amino acids or nucleic acids appear to obtain the number of matching locations, dividing the number of matching locations by the total number of locations in the comparable regions (that is, the size of a range), and multiplying a result of the division by 100 to obtain the percentage of the sequence identity. The percentage of the sequence identity may be determined using a known sequence comparison program, for example, BLASTN (NCBI), BLASTP (NCBI), CLC Main Workbench (CLC bio), or MegAlign™ (DNASTAR Inc). Unless otherwise mentioned in the specification, the selection of the parameters used to execute the program may be as follows: Ktuple=2, Gap Penalty=4, and Gap length penalty=12.

Various levels of sequence identity may be used to identify various types of polypeptides or polynucleotides having the same or similar functions or activities. For example, the sequence identity may include a sequence identity of about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, about 99% or more, or 100%.

The term “genetic modification” as used herein may refer to an artificial alteration in a constitution or structure of a genetic material of a cell.

The symbol “%” as used herein indicates w/w %, unless otherwise mentioned.

An aspect of an embodiment provides a recombinant microorganism having a genetic modification that increases a dehalogenase activity as compared to the same microorganism, wherein the microorganism has an increased dehalogenase activity as compared with a parent strain.

In an embodiment, the recombinant microorganism may be a type of enzyme that catalyzes the removal of a chlorine atom, a bromine atom, or an iodine atom from a dehalogenase substrate. A dehalogenase may be a haloalkane dehalogenase, an alkalihalidase, an (S)-2-haloacid dehalogenase, an (R)-2-haloacid dehalogenase, or a combination thereof

A protein having a dehalogenase activity may have a sequence identity of 50% or more, 55% or more, 60% or more, 65% or more, 70% or more, 75% or more, 80% or more, 85% or more, 90% or more, 95% or more, 96% or more, 97% or more, 98% or more, 99% or more, or 100% with respect to an amino acid sequence of SEQ ID NO: 1 (dhlA), 3 (BC3334N122Y), or 5(hdl4a). The protein having the dehalogenase activity may have an amino acid sequence of SEQ ID NO: 1, 3, or 5. The protein having an amino acid sequence of SEQ ID NO: 1 may be classified as a haloalkane dehalogenase. The protein having an amino acid sequence of SEQ ID NO: 1 may be an enzyme using 1-haloalkane and water as substrates to catalyze a reaction for the production of primary alcohols and halides. The protein having the dehalogenase activity may be an enzyme belonging to EC 3.8.1.5, and such a dehalogenase may be classified as an (S)-2-haloacid dehalogenase. The (S)-2-haloacid dehalogenase protein may be an enzyme using (S)-2-haloacid and water as substrates to catalyze a reaction for the production of (R)-hydroxy acids and halides. The protein having the (S)-2-haloacid dehalogenase activity may be an enzyme belonging to EC 3.8.1.2, and such a dehalogenase may be a Bacillus cereus BC3334 protein (SEQ ID NO: 17) or a mutant thereof. The mutant may have an N122Y substitution (SEQ ID NO: 3), or the dehalogenase may be a hdl4a protein (SEQ ID NO: 5).

The genetic modification may include an increase in expression of a gene encoding a protein having a dehalogenase activity. The genetic modification may include an increase in the copy number of the gene. The recombinant microorganism may include at least one foreign gene having an increased copy number.

Regarding the recombinant microorganism, the gene may be introduced to a microorganism by methods known in the art, such as transformation and electroporation.

The at least one foreign gene encoding the protein having the dehalogenase activity may have a nucleotide sequence of SEQ ID NO: 2, 4, or 6. In addition, such a gene may be codon-optimized by a host cell which is the recombinant microorganism. The term “codon-optimized gene” as used herein refers to a gene that encodes the same amino acid as a gene that is not codon-optimized, but at least one of endogenous codons of the gene is substituted with a codon advantageous for expression in a corresponding host. The nucleotide sequences of SEQ ID NOs: 2, 4, and 6 are genes encoding haloalkane dehalogenase (dhlA) derived from Xanthobacter autotrophicus, a BC 3334 N122Y mutant having an N122Y substitution in BC 3334 derived from Bacillus cereus, and hdl4a derived from Pseudomonas saitens (KCTC 13107BP), respectively.

The recombinant microorganism may belong to the genus Pseudomonas, Xanthobacter, Escherichia, Agrobacterium, Corynebacterium, Rhodococcus, Mycobacterium, or Klebsiella. The genus Pseudomonas may include P. saitens (KCTC 13107BP). The genus Escherichia may be E. coli. The genus Xanthobacter may include X. autotrophicus.

The microorganism may include at least one (for example, 2 or more, 3 or more, 4 or more, 5 or more, 10 or more, or 50 or more) foreign genes encoding the protein having the dehalogenase activity. When the microorganism includes a plurality of genes, the plurality of genes may be different from each other or they may include multiple copies of the same gene. These genes may be integrated into the genome of the microorganism, or may be independent of the genome.

The recombinant microorganism may reduce a concentration of a fluorine-containing compound represented by Formula 1 or Formula 2 in a sample:

C(R₁)(R₂)(R₃)(R₄) <Formula 1>

(R₅)(R₆)(R₇)C—[C(R₁₁)(R₁₂)]n-C(R₈)(R₉)(R₁₀). <Formula 2>

In Formula 1 and 2,

n may be an integer from 0 to 10,

R₁, R₂, R₃, and R₄ may each independently be fluorine (F), chlorine (CI), bromine (Br), iodine (I), or hydrogen (H), wherein at least one selected from R₁, R₂, R₃, and R₄ may be F, and

R₅, R₆, R₇, R₈, R₉, R₁₀, R₁₁, and R₁₂ may each independently be F, Cl, Br, I, or H, wherein at least one selected from R₅, R₆, R₇, R₈, R₉, R₁₀, R₁₁, and R₁₂ may be F.

The reduced concentration may be achieved by introduction of a hydroxyl group to a carbon by the protein through an action on a C—F bond or a C—H bond of the fluorine-containing compound, or by accumulation of the fluorine-containing compound in a cell of the microorganism. In addition, the reduced concentration may be achieved by cleavage of a C—F bond of the fluorine-containing compound, conversion of the fluorine-containing compound into a different substrate, or accumulation of the fluorine-containing compound in a cell. The sample may be a liquid sample or a gaseous sample. The sample may be industrial sewage or waste gas. Any material including the fluorine-containing compound may be used as the sample. The fluorine-containing compound may be CF₄, CHF₃, CH₂F₂, CH₃F, or a mixture thereof.

Another aspect of an embodiment provides a composition for use in reducing a concentration of a fluorine-containing compound represented by Formula 1 or Formula 2 in a sample, the composition including a recombinant microorganism having a genetic modification that increases a dehalogenase activity as compared to a parent strain.

Regarding the composition, the recombinant microorganism, the sample, and the fluorine-containing compound are the same as described above.

The term “reduced” as used herein in connection with the composition refers to a reduction in the concentration of the fluorine-containing compound in the sample, and may include a complete removal of the fluorine-containing compound in the sample. The sample may be a liquid sample or a gaseous sample. The sample can be free of the microorganism, or the microorganism may be present therein. The composition may further include a substance that increases the solubility of the fluorine-containing compound in a medium or a culture.

The reduced concentration of the fluorine-containing compound may be achieved by contacting the composition with the sample. The contacting may be performed in a liquid or solid phase. The contacting may be performed, for example, by contacting the sample with a culture of the microorganism in a medium. The culturing may be performed under conditions where the microorganism may proliferate.

Another aspect of an embodiment provides a method of reducing a concentration of a fluorine-containing compound in a sample, the method including contacting a sample including a fluorine-containing compound represented by Formula 1 or Formula 2 with a recombinant microorganism having a genetic modification that increases a dehalogenase activity as compared to a parent strain, so as to reduce the concentration of the fluorine-containing compound in the sample.

Regarding the method, the recombinant microorganism and the sample including the fluorine-containing compound are the sample as described above.

Regarding the method, the contacting may be performed in a liquid phase or a gaseous phase. The contacting may be performed, for example, by contacting the sample with the cultured microorganism in a medium. The culturing may be performed under conditions where the microorganism may proliferate. The contacting may be performed in a closed container. The contacting may be performed when a growth stage of microorganism is in an exponential phase or a stationary phase. The culturing may be performed under aerobic or anaerobic conditions. The contacting may be performed under conditions where the recombinant microorganism may survive in the closed container. Such viable conditions may include conditions where the recombinant microorganism may proliferate or conditions where the recombinant microorganism may be allowed to be in a resting state.

Regarding the method, the sample may be a liquid sample or a gaseous sample. The sample may be industrial sewage or waste gas. The sample may contact the culture of the microorganism not only in a passive manner, but also in an active manner. The sample may be, for example, sparged in a culture medium of the microorganism. That is, the sample may be blown through a medium or a culture medium. Here, the sparging may be blowing of the sample from the bottom to the top of the medium or the culture medium. The sparging may be performed via injecting of droplets of the sample.

Regarding the method, the contacting may be performed in a batch or continuous manner. The contacting may include, for example, contacting the resulting sample from the contacting above with a new recombinant microorganism having a genetic modification that increases a dehalogenase activity as compared to a parent strain. The contacting with the new recombinant microorganism may be performed twice or more, for example, twice, three times, five times, or ten times or more. The contacting may be continued or repeated until the concentration of the fluorine-containing compound reaches a desired reduced concentration.

Another aspect of an embodiment provides a method of preparing a recombinant microorganism having an increased dehalogenase activity, the method including introducing a gene to a microorganism, Pseudomonas saitens (KCTC 13107BP), wherein the gene includes at least one selected from the group consisting of a gene encoding a polypeptide (BC3334 N122Y) having a sequence identity of 85% or more with respect to an amino acid sequence of SEQ ID NO: 3, and a gene encoding a polypeptide (hdl4a) having a sequence identity of 85% or more with respect to an amino acid sequence of SEQ ID NO: 5.

The recombinant microorganism according to an aspect of an embodiment may be used for the removal of the fluorine-containing compound in the sample.

The composition according to an aspect of another embodiment may be used for the reduction in the concentration of the fluorine-containing compound in the sample.

The method of reducing the concentration of the fluorine-containing compound in the sample according to an aspect of another embodiment may effectively reduce the concentration of the fluorine-containing compound in the sample.

Hereinafter, the present invention will be described in more detail with reference to Examples. However, these Examples are provided for illustrative purposes only, and the invention is not intended to be limited by these Examples.

### Example 1: Decomposition of Fluorine-Containing Compound by Pseudomonas Saitens Including Dehalogenase Introduced Thereto

A microorganism capable of reducing a concentration of CF₄ in industrial sewage was selected. As a result, a strain of P. saitens capable of decomposing CF₄ was selected.

In detail, the sludge of the wastewater discharged from the plant of Samsung Electronics (Giheung, Korea) was smeared on an agar plate including a carbon-free medium (an agar medium supplemented with 0.7 g/L of K₂HPO₄, 0.7 g/L of MgSO₄.7H₂O, 0.5 g/L of (NH₄)₂SO₄, 0.5 g/L of NaNO₃, 0.005 g/L of NaCl, 0.002 g/L of FeSO₄.7H₂O, 0.002 g/L of ZnSO₄.7H₂O, 0.001 g/L of MnSO₄, and 15 g/L of agar), and the agar plate was added to a GasPak™ Jar (BD Medical Technology). The inside of the jar was filled with 99.9 v/v % CF₄, and the jar was sealed for the standing culture at a temperature of 30□ under anaerobic conditions. Single colonies formed after the culturing were cultured using a high throughput screening (HTS) system (Thermo Scientific/Liconic/Perkin Elmer). Each cultured single colony was inoculated on a 96-well microplate containing 100 μL of medium per well, and then, was subjected to static culture at a temperature of about 30□ for 72 hours under aerobic conditions. Meanwhile, the growth ability of the colonies was observed by measuring the absorbance at 600 nm every 12 hours. The LB medium used herein included 10 g/L of tryptone, 5 g/L of yeast extract, and 10 g/L of NaCl.

The top 2% of strains showing excellent growth ability were selected and were each inoculated in a glass serum bottle (volume of 75 mL) containing 10 mL of the LB medium so as to have an OD600 of 0.5. The glass serum bottle was sealed, and then, CF₄ was injected thereto by using a syringe so as to have 1,000 ppm of CF₄ gas. The glass serum bottle was incubated in a shaking incubator for 4 days at a temperature of 30□ while be stirred at a speed of 230 rpm, and then, the amount of CF₄ in a head space was analyzed.

For the analysis, 0.5 ml of CF₄ contents was collected by using a syringe from the head space, and then, was injected into a gas chromatograph (GC, Agilent 7890, Palo Alto, Calif., USA). The injected CF₄ was separated by a CP-PoraBOND Q column (25 m length, 0.32 mm i.d., 5 um film thickness, Agilent) and changes in the concentration of the CF₄ was analyzed by MSD (Agilent 5973, Palo Alto, Calif., USA). Helium was used as carrier gas, and was flowed into the column at a rate of 1.5 ml/min. Regarding conditions for the GC, a temperature at an inlet was 250□, and an initial temperature of 40□ was maintained for 2 minutes and raised up to 290□ at a speed of 20□/min. Regarding conditions for the MS, an ionization energy was 70 eV, an interface temperature was 280□, an ion source temperature was 230□, and a quadrupole temperature was 150□. As a control group, 1,000 ppm of CF₄ containing no cells was measured after incubation under the same conditions.

Consequently, compared to a control group having no cells, the concentration of CF₄ was reduced by 10.4% in the separated microorganisms. The microorganisms had decomposition activity of 0.005 umol/g-cell/min. To identify the selected strains, a 16s rRNA gene was amplified using the genome of separated cells as a template. A nucleotide sequence of the 16s rRNA gene was then analyzed according to BLAST Assembled Genomes.

A genome obtained by the assembling segments had a final size of 5.1 Mb, and a G+C content was 59.14%. As a result of automatic annotation using Prokaryotic Genome Annotation Pipeline, a total of 328 genes, 25 rRNA operons, 73 tRNAs, and 1 tmRNA were present in the genome. In addition, as a result phylogenetic tree analysis, it was confirmed that the microorganisms belonged to the genus Pseudomonas. However, there was no species having a perfect sequence identity with respect to a known species belonging to the genus Pseudomonas.

The separated microorganism was newly named as Pseudomonas saitens (hereinafter, referred to as “SF1”), deposited at the Korean Collection for Type Culture (KCTC), which is an international depository authority under the Budapest Treaty, on Sep. 12, 2016, and assigned the accession number of KCTC13107BP. The SF1 microorganism was then further modified as described below.

1. Amplification of Dehalogenase dhlA Gene Derived from Xanthobacter Autotrophicus

A haloalkane dehalogenase (dhlA) of X. autotrophicus GJ10 was selected as an enzyme having activity to decompose a fluorine-containing hydrocarbon.

A gene (SEQ ID NO: 2) encoding the dhlA was amplified by PCR performed thereon using primer sets of SEQ ID Nos: 7 and 8 as a primer and X. autotrophicus GJ10 genome as a template.

The amplified PCR products were ligated to a pBBR122 vector that was amplified by PCR performed thereon using a pBBR122 vector (MoBiTec) as a template and primer sets of SEQ ID Nos: 15 and 16 as a primer, and then, inserted to a chloramphenicol ORF region, thereby obtaining a pB_dhlA vector for expression of the dhlA. The vector was introduced into an SF1 microorganism via electroporation, and whether the introduction was successful or not was confirmed by antibiotic resistance. The SF1 microorganism to which the dhlA was introduced was designated as SF1-pB-dhlA.

2. Amplification of Dehalogenase hdl4a Gene Derived from Pseudomonas Saitens

A gene (SEQ ID NO: 6) encoding haloacid dehalogenase (hdl4a) was amplified by PCR performed thereon using sequences of SEQ ID Nos: 9 and 10 as a primer set and the SF1 genome as a template.

The amplified PCR products were connected to a pBBR122 vector that was amplified by PCR performed thereon using a pBBR122 vector (MoBiTec) as a template and sequences of SEQ ID Nos: 15 and 16 as a primer set, and then, inserted to a chloramphenicol ORF region, thereby obtaining a pB_hdl4a vector for expression of the hdl4a.

The vector was introduced into an SF1 microorganism via electroporation, and whether the introduction was successful or not was confirmed by antibiotic resistance. The SF1 microorganism to which the hdl4a SF1 was introduced was designated as SF1-pB-hdl4a.

3. Preparation of BC3334 N122Y Gene Derived from Bacillus cereus

(1) Amplification of Haloacid Dehalogenase Gene (BC3334) Derived from Bacillus cereus and Introduction of Gene to E. coli

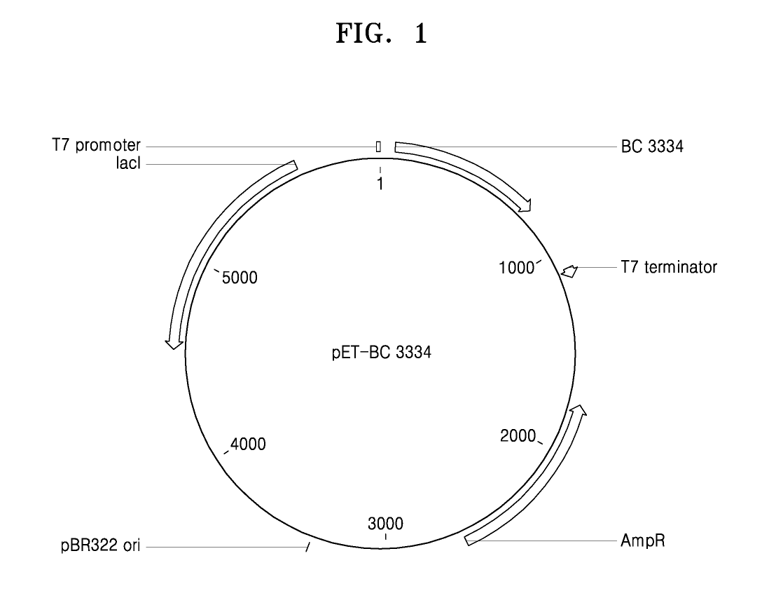

B. cereus (KCTC 3624) was cultured overnight in an LB medium while being stirred at a temperature of 30□ at a speed of 230 rpm, and then, genomic DNA was isolated using the total DNA extraction kit (Invitrogen Biotechnology). PCR was performed using this genomic DNA as a template and a set of primers having nucleotide sequences to amplify and obtain a BC3334 gene. The amplified BC3334 gene was ligated with pETDuet-1 vector (Novagen, Cat. No. 71146-3), which was digested with restriction enzymes, NcoI and HindIII, using the InFusion Cloning Kit (Clontech Laboratories, Inc.), thereby preparing a pET-BC3334 vector. FIG. 1 is a vector map of the pET-BC334 vector. The BC3334 has an amino acid sequence of SEQ ID NO: 17, and its gene has a nucleotide sequence of SEQ ID NO: 18.

Next, the prepared pET-BC3334 vector was introduced to E. coli by a heat shock method, and then, cultured in an LB plate containing 100 μg/mL of ampicillin. Strains showing ampicillin resistance were selected. Then, a finally selected strain was designated as a recombinant E. coli BL21/pET-BC3334 wt.

(2) Recombinant E. coli Expressing Mutant BC3334 Gene

In this section, mutants were prepared to improve the activity of removing the fluorine-containing compound in the sample by BC3334. Asparagine (hereinafter, referred to as “N122”) at position 122 of the amino acid sequence of SEQ ID NO: 17 was substituted with tyrosine. A gene encoding the mutants was introduced to E. coli, and its activity of removing CF₄ in a sample was examined.

The preparation of the N122Y mutants of SEQ ID NO: 17 was performed using a QuikChange II Site-Directed Mutagenesis Kit (Agilent Technology, USA). Site-directed mutagenesis using the kit was performed using PfuUlta high-fidelity (HF) DNA polymerase for mutagenic primer-directed replication of both plasmid strands with the highest fidelity. The basic procedure utilized a supercoiled double-stranded DNA (dsDNA) vector with an insert of interest and two synthetic oligonucleotide primers, both containing the desired mutation. The oligonucleotide primers, each complementary to opposite strands of the vector, were extended during temperature cycling by PfuUltra HF DNA polymerase, without primer displacement. Extension of the oligonucleotide primers generated a mutated plasmid containing staggered nicks. Following temperature cycling, the product was treated with DpnI. The DpnI endonuclease (target sequence: 5′-Gm⁶ATC-3′) was specific for methylated and hemimethylated DNA, and was used to digest the parental DNA template and to select for mutation-containing synthesized DNA. The nicked vector DNA incorporating the desired mutations was then transformed into XL1-Blue supercompetent cells.

In detail, PCR was performed using the pET28a-BC3334 wt vector prepared in (1) as a template and the primer sets for the BC3334 N122Y mutants as a primer and PfuUlta HF DNA polymerase to obtain mutated vectors containing staggered nicks. These vector products were treated with DpnI to select mutation-containing synthesized DNAs. The nicked vector DNA incorporating the desired mutations was then transformed into XL1-Blue supercompetent cells, thereby cloning a pET28a-BC3334 N122Y vector.

Lastly, the cloned pET28a-BC3334 N122Y vector was introduced to a strain of E. coli BL21 in the same manner as in (1), and a finally selected strain was designated as a recombinant E. coli BL21/pET28a-BC3334 N122Y.

A BC3334 N122Y gene (SEQ ID NO: 4) was amplified by PCR performed thereon using the genome of BL21/pET28a-BC3334 N122Y as a template and primer sets of SEQ ID Nos: 13 and 14 as a primer.

The amplified PCR products were ligated to a pBBR122 vector that was amplified by PCR performed thereon using a pBBR122 vector (MoBiTec) as a template and primer sets of SEQ ID Nos: 15 and 16 as a primer, and then, inserted to a chloramphenicol ORF region, thereby obtaining a pB_BC3334 N122Y vector for expression of the BC3334 N122Y. The vector was introduced into an SF1 microorganism via electroporation, and whether the introduction was successful or not was confirmed by antibiotic resistance. The SF1 microorganism into which the BC3334 N122Y was designated as SF1-pB-BC3334 N122Y.

4. Decomposition of Fluorine-Containing Compound by Circulation Process

It was examined whether the SF1_dhlA, the hdl4a, and the BC3334 N122Y prepared in Sections (1) to (3) affects the reduction of the fluorine-containing compound in a sample through contacting the sample with the fluorine-containing compound.

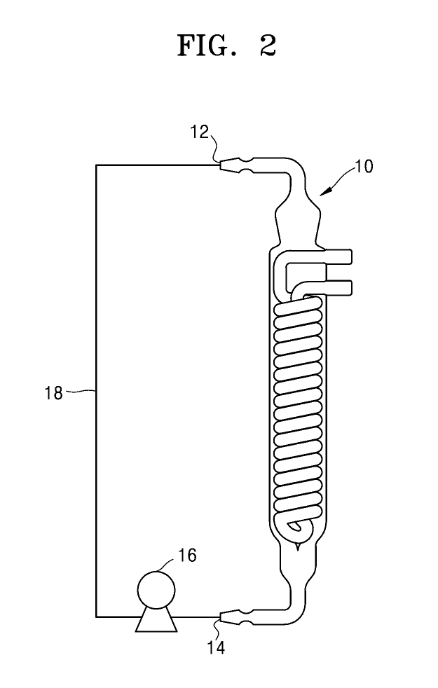

As shown in FIG. 2, 50 ml of an LB medium and 1,000 ppm of CF₄ gas were added to a glass Dimroth coil reflux condenser (a reactor length: 350 mm, an exterior diameter: 35 mm, and an interior volume: 200 mL) that was sterilized and vertically oriented, and then, the LB medium was subjected to circulation. FIG. 2 is a schematic diagram of the glass Dimroth reflux condenser (10). The LB medium was supplied to an inlet (12) of an upper portion of the condenser (10), flowed through an inner wall of the condenser (10), and discharged to an outlet (14) of a lower portion of the condenser (10). The discharged LB medium was re-supplied to the inlet (12) along a circulation line (18). Although not shown in FIG. 2, to maintain a temperature of the condenser (10), an inner screwed pipe of the condenser (10) was connected to a constant temperature bath having a temperature of 30□. The circulation is performed by a pump (16). Here, the circulation rate of the LB medium was maintained at 4 mL/min. After an appointed period of time, i.e., 0, 42, 90, and 142 hours, the amount of the CF₄ gas in the condenser was confirmed by gas chromatography mass-spectrum (GC-MS). Then, it was confirmed that there was no change in the amount of CF₄ gas.

Subsequently, the recombinant microorganisms of sections (1) to (3) and the control group were each inoculated on an LB medium in the condenser by using a syringe, so as to have an initial concentration of 5.0 on the basis of OD600. The control group included a strain of the same microorganism as the test groups, but the stain included an empty vector introduced thereto. That is, the control group was recombinant E. coli BL21/pET28a. The circulation rate of the LB culture was 4 mL/min, and the temperature inside the condenser was maintained at 30□. After the strain inoculation and the elapse of 0, 42, 90, and 142 hours, the amount of CF₄ gas in the condenser was confirmed by GC-MS. Then, the decomposition rate of CF₄ was calculated according to Equation 1, and the results are shown in Table 1.

Decomposition rate of CF₄=[(Initial amount of CF₄−amount of CF₄ after reaction)/initial amount of CF₄]×100 <Equation 1>

TABLE 1

| | Decomposition rate of CF₄ (%) | | | |
|---|---|---|---|---|
| Strain | 0 hour | 42 hours | 90 hours | 142 hours |
| SF1-vec (control group) | 0 | 12.4 | 21.0 | 21.9 |
| SF1-hdl4a | 0 | 21.5 | 27.7 | 28.7 |
| SF1-dhlA | 0 | 6.8 | 29.7 | 29.3 |
| SF1-BC3334 N122Y | 0 | 17.0 | 26.6 | 31.9 |

As shown in Table 1, after 142 hours of the culture, the three recombinant microorganisms showed high removal rates for the fluorine-containing compounds, compared to the control group.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant Pseudomonas saitens microorganism comprising a genetic modification that increases dehalogenase activity as compared to a parent strain Pseudomonas saitens KCTC 13107BP, wherein the dehalogenase activity comprises the activity of at least one dehalogenase selected from the group consisting of a haloalkane dehalogenase, a polypeptide having a sequence identity of 85% or more with respect to an amino acid sequence of SEQ ID NO: 3, and a polypeptide having a sequence identity of 85% or more with respect to an amino acid sequence of SEQ ID NO: 5.

2. The recombinant microorganism of claim 1, wherein the genetic modification increases expression of a gene encoding the dehalogenase.

3. The recombinant microorganism of claim 1, wherein the genetic modification increases a copy number of a gene encoding the dehalogenase.

4. The recombinant microorganism of claim 1, wherein the haloalkane dehalogenase is an enzyme belonging to EC 3.8.1.5.

5. The recombinant microorganism of claim 1, wherein haloalkane dehalogenase has a sequence identity of 85% or more with respect to an amino acid sequence of SEQ ID NO: 1.

6. A method of reducing a concentration of a fluorine-containing compound in a sample, the method comprising:
contacting a recombinant microorganism of claim 1 with a sample comprising a fluorine-containing compound represented by Formula 1 or Formula 2, so as to reduce the concentration of the fluorine-containing compound in the sample:
C(R₁)(R₂)(R₃)(R₄) <Formula 1>
(R₅)(R₆)(R₇)C—[C(R₁₁)(R₁₂)]n-C(R₈)(R₉)(R₁₀) <Formula 2>
wherein, in Formulae 1 and 2,
n is an integer from 0 to 10,
R₁, R₂, R₃, and R₄ are each independently fluorine (F), chlorine (CI), bromine (Br), iodine (I), or hydrogen (H), and at least one selected from R₁, R₂, R₃, and R₄ is F, and
R₅, R₆, R₇, R₈, R₉, R₁₀, R₁₁, and R₁₂ are each independently F, Cl, Br, I, or H, and at least one selected from R₅, R₆, R₇, R₈, R₉, R₁₀, R₁₁, and R₁₂ is F.

7. The method of claim 6, wherein the genetic modification increases expression of a gene encoding the dehalogenase.

8. The method of claim 6, wherein the genetic modification increases a copy number of a gene encoding the dehalogenase.

9. The method of claim 6, wherein the haloalkane dehalogenase is an enzyme belonging to EC 3.8.1.5.

10. The method of claim 6, wherein the haloalkane dehalogenase has a sequence identity of 85% or more with respect to an amino acid sequence of SEQ ID NO: 1.

11. The method of claim 6, wherein the contacting is performed in a closed container.

12. The method of claim 6, wherein the contacting comprises culturing or incubating the recombinant microorganism in contact with the sample.

13. The method of claim 6, wherein the contacting comprises culturing the recombinant microorganism under conditions in which the recombinant microorganism proliferates in a closed container.

14. The method of claim 6, wherein the fluorine-containing compound is CF₄, CHF₃, or CH₂F₂.

15. The method of claim 6, wherein the recombinant microorganism cleaves a C—F bond of the fluorine-containing compound, converts the fluorine-containing compound into a different substance, or accumulates the fluorine-containing compound in the recombinant microorganism, to reduce the concentration of the fluorine-containing compound in the sample.

16. The method of claim 6, wherein the sample is a liquid or gas.

17. A method of preparing a recombinant microorganism of claim 1 having an increased dehalogenase activity, the method comprising
introducing into a Pseudomonas saitens KCTC 13107BP microorganisma gene encoding a haloalkane dehalogenase, a gene encoding a polypeptide (BC3334 N122Y) having a sequence identity of 85% or more with respect to an amino acid sequence of SEQ ID NO: 3, a gene encoding a polypeptide (hdl4a) having a sequence identity of 85% or more with respect to an amino acid sequence of SEQ ID NO: 5, or a combination thereof.
